(19) United States
(12) Patent Application Publication
(10) Pub. No.: US 2026/0241204 A1
(43) Pub. Date: Aug. 20, 2026

(54) METHOD FOR PLANNING RADIOTHERAPY TREATMENT

(71) Applicant: Elekta Instrument AB, Stockholm (SE)

(72) Inventors: Håkan Nordström, Stockholm (SE); Joakim Sebastian da Silva, Stockholm (SE)

(21) Appl. No.: 19/543,289

(22) Filed: Feb. 18, 2026

(30) Foreign Application Priority Data
Feb. 20, 2025 (GB) 2502502.4

(51) Int. Cl. A61N 5/10

(52) U.S. Cl. CPC A61N 5/1031; A61N 5/1084

(57) ABSTRACT

A method for planning radiotherapy treatment in a treatment volume including multiple discrete treatment targets. The method comprises: identifying the plurality of treatment targets in the treatment volume; defining a set of treatment objectives for the treatment volume; and separating the plurality of treatment targets into at least a first group and a second group. Each group comprises one or more treatment targets for which a separation therebetween is below a threshold amount. Each treatment target in the first group is separated from each treatment target in the second group by at least the threshold amount. The method further comprises: inputting the first group of treatment targets and the treatment objectives into a first optimisation program to generate a first set of treatment parameters; inputting the second group of treatment targets and the treatment objectives into a second optimisation program to generate a second set of treatment parameters; and inputting the first and second groups of treatment targets, the first and second sets of treatment parameters, and the treatment objectives into a third optimisation program to generate a third set of treatment parameters.

### CLAIM FOR PRIORITY

This application claims the benefit of priority of British Application No. 2502502.4, filed Feb. 20, 2025, which is hereby incorporated by reference in its entirety.

### TECHNICAL FIELD

The present disclosure relates to a method for planning radiotherapy treatment in a treatment volume including multiple discrete treatment targets, and to a computer-readable medium for implementing such a method.

### BACKGROUND

The development of surgical techniques has made great progress over the years. For instance, patients in need of brain surgery may instead have non-invasive surgery which drastically reduces the trauma to the patients, by irradiating regions of tissue to be treated, thereby eliminating the need for making incisions in the skull and through healthy brain tissue.

One system for non-invasive surgery is the Leksell Gamma Knife® Perfexion system, which provides such surgery by means of gamma radiation. Other example systems include the Leksell Gamma Knife Icon, and Elekta Esprit. This is not an exhaustive list. In such systems, the radiation is emitted from a large number of radioactive sources, or from a single or small number of moveable radioactive sources or radiation sources, and is focused into beams by means of collimators, i.e. passages or channels for obtaining a beam of limited cross section, towards a defined target or treatment volume. Each beam provides a dose of gamma radiation which, by itself, is insufficient to treat intervening tissue. However, a therapeutic dose occurs where a plurality of radiation beams intersect or converge, causing the radiation to reach therapeutic levels. The point of convergence is hereinafter referred to as the “focus point”. Schematic illustrations of such a system are provided in FIGS. 1a, 1b and 1c, and are described in further detail in the detailed description section of this document.

In order to effectively irradiate target tissue (referred to herein as treatment targets, which may be cancerous or otherwise unhealthy tissue) within a treatment volume such as the brain, and protect healthy tissue surrounding the treatment targets, the radiotherapy treatment must be carefully planned. Errors in treatment planning can cause unwanted irradiation of healthy tissue, and can reduce the dose received by treatment targets, thereby reducing the efficacy of the treatment.

One approach for treatment planning is inverse treatment planning, which uses treatment optimisation. In treatment optimisation, the desired treatment outcomes for the treatment are first defined. Then, based on the desired treatment outcomes, the treatment planning performs computational modelling (referred to herein as an optimisation algorithm, or an optimisation problem) which is configured to optimise treatment parameters of the radiotherapy system in order to achieve the desired treatment outcomes. Because the treatment optimisation is configured to work out the treatment parameters (system inputs) which best achieve the desired treatment outcomes (system outputs), it enables inverse treatment planning. In effect, inverse treatment planning starts with the desired treatment outcomes (desired output), and works backwards from that to find the best (i.e. optimised) treatment parameters (inputs). This is in contrast to manual planning, in which the treatment parameters (inputs) would be iteratively adjusted on a trial-and-error basis until the desired treatment outcomes are achieved.

The desired treatment outcomes include the locations of treatment targets to be irradiated in the treatment volume, and treatment objectives for the treatment targets and surrounding (e.g. healthy) tissue, which can include hard and soft constraints. These may be input by a surgeon. However, in some examples, the treatment objectives may be based on pre-defined clinical protocols rather than being input by a surgeon. The surgeon may define the location(s) of the treatment target(s) based on a scan of the treatment volume (such as an MRI scan, an X-ray scan, or a CT scan), and may in some examples also define location(s) of organ(s) at risk in the treatment volume.

Optimisation algorithms for use in inverse planning optimisation are known and already used. Examples are described in the following literature:
US2018/311509
US2019/255355
“A linear programming approach to inverse planning in Gamma Knife radiosurgery” by J. Sjölund et al., Med Phys. 46 (4) April 2019 (pages 1533-1544)

For each treatment target, the treatment parameters of the radiotherapy system may include a number of irradiations, a duration of each irradiation, a beam diameter of each irradiation, and a beam shape of each irradiation. Where there is a plurality of treatment targets, the treatment parameters must be defined for each treatment target.

The optimisation problem is computationally complex and burdensome, because for each treatment target, there are many treatment parameters to control and optimise. In effect, there are many variables (sometimes referred to herein as degrees of freedom) to optimise. Furthermore, because each irradiation will impart a radiation dose not only to the treatment target but also to nearby treatment target and to the healthy tissue through which it passes, the complexity of the optimisation problem increases rapidly as the number of treatment targets to be irradiated, and as the number of required irradiations of each treatment target, increases. As a result, treatment planning can be a slow and computationally burdensome task. Where the number of treatment targets is very high, the complexity may be so high that inverse treatment planning is not practicably possible.

Therefore, there is a demand for improvements to existing treatment optimisation approaches, which enable inverse treatment planning to be performed quickly and efficiently even where there is a large number of treatment targets. For example, where a patient has a large number of cancerous metastases, the number of treatment targets may be large, thereby necessitating improvements to optimisation in order for inverse planning to be possible.

### SUMMARY

The subject matter of the present disclosure has been developed in order to address or reduce the problems described above.

The inventors have found that treatment planning time increases non-linearly as the number of treatment targets increases. The inventors have also found that where a treatment volume includes a group of closely clustered treatment targets, the treatment targets are highly influencing on one another and on the intervening healthy tissue from a radiation dosage perspective. In particular, a radiation dose received by one treatment target in the cluster will contribute to the dosage of radiation received by adjacent treatment targets in the cluster and will contribute to the dosage of radiation received by intervening healthy tissue, such that all treatment targets in the cluster must be considered together, in a single optimisation problem, to ensure high plan quality and that adverse clinical effects do not occur. However, advantageously, the inventors have also found that where there is another group of closely clustered treatment targets, which is relatively isolated from the second group, the radiation doses received by treatment targets of the first group may have little or no effect on radiation doses received by treatment targets or healthy tissue in the second group. In this case, it may not be necessary to include the treatment targets of the first group in the same optimisation problem as the treatment targets of the second group. In this case, the treatment parameters for the first group of treatment targets can be optimised in a first optimisation problem, and the treatment parameters for the second group of treatment targets can be optimised in a second optimisation problem. Because optimisation time increases non-linearly with each additional treatment target, and because the total number of treatment targets in each group is less than the total number of treatment targets, optimising the treatment parameters for the two groups in separate respective optimisation problems will therefore significantly reduce the total optimisation time.

An example optimisation problem approach according to the present disclosure is described under the sub-heading “Example optimisation problem” towards the end of the “Detailed description” section of this document. More detail on such an optimisation problem is provided in US2018/311509, US2019/255355, and J. Sjölund et al., the contents of which are incorporated into the present disclosure by reference.

Accordingly, at its most general, the present invention provides an optimisation method in which treatment targets are split into groups, where each group has little or no radiation effect on adjacent groups, and then separately solving an optimisation problem for each group. Therefore, known optimisation problems (for example, those described in US2018/311509, US2019/255355, and J. Sjölund et al.) can be used to achieve treatment planning for multiple treatment targets more quickly and computationally efficiently than was previously possible. These three disclosures are incorporated into the present disclosure by reference.

In a general aspect, there is provided a method for planning radiotherapy treatment in a treatment volume including multiple discrete treatment targets, the method comprising:
identifying the plurality of treatment targets in the treatment volume;
defining a set of treatment objectives for the treatment volume;
separating the plurality of treatment targets into at least a first group and a second group, wherein each group comprises one or more or more treatment targets for which a separation therebetween is below a threshold amount, and wherein each treatment target in the first group is separated from each treatment target in the second group by at least the threshold amount;
inputting the first group of treatment targets and the treatment objectives into a first optimisation program to generate a first set of treatment parameters; and
inputting the second group of treatment targets and the treatment objectives into a second optimisation program to generate a second set of treatment parameters.

In a first claimed aspect, there is provided a method for planning radiotherapy treatment in a treatment volume including multiple discrete treatment targets, the method comprising:
identifying the plurality of treatment targets in the treatment volume;
defining a set of treatment objectives for the treatment volume;
separating the plurality of treatment targets into at least a first group and a second group, wherein each group comprises one or more or more treatment targets for which a separation therebetween is below a threshold amount, and wherein each treatment target in the first group is separated from each treatment target in the second group by at least the threshold amount;
inputting the first group of treatment targets and the treatment objectives into a first optimisation program to generate a first set of treatment parameters;
inputting the second group of treatment targets and the treatment objectives into a second optimisation program to generate a second set of treatment parameters; and
inputting the first and second groups of treatment targets, the first and second sets of treatment parameters, and the treatment objectives into a third optimisation program to generate a third set of treatment parameters.

In the first aspect, the third optimisation problem may provide a final sweep of all treatment targets, to check that there are no unwanted effects when all treatment targets are treated in a single session. The third optimisation problem may therefore fine-tune the treatment parameters to reduce or eliminate any such effects.

In a second claimed aspect, there is provided a method for planning radiotherapy treatment in a treatment volume including multiple discrete treatment targets, the method comprising:
identifying the plurality of treatment targets in the treatment volume;
defining a set of treatment objectives for the treatment volume;
separating the plurality of treatment targets into at least a first group and a second group, wherein each group comprises one or more treatment targets for which a separation therebetween is below a threshold amount, and wherein each treatment target in the first group is separated from each treatment target in the second group by at least the threshold amount;
inputting the first group of treatment targets and the treatment objectives into a first optimisation program to generate a first set of treatment parameters;
inputting the second group of treatment targets and the treatment objectives into a second optimisation program to generate a second set of treatment parameters;
wherein the first and second optimisation programs run in parallel to generate the first and second sets of treatment parameters.

In the second aspect, running the first and second optimisation problems in parallel may further reduce optimisation time, because in this case the total elapsed time for the optimisation would not be the sum of the two respective optimisation times.

The first and second aspects may be combined. That is to say, the first and second optimisation problems may be performed in parallel, and the third optimisation problem may then be performed. However, in the second aspect, the third optimisation may alternatively be performed after the first and second optimisation problems have been sequentially performed.

Each optimisation program is configured to execute an optimisation problem (sometimes referred to herein as an optimisation algorithm) according to the present disclosure.

In some examples, at least one of the groups may include two or more treatment targets. In other examples, each group may include two or more treatment targets. Furthermore, more than two groups may be defined, each comprising one or more (for example two or more) treatment targets. Where there are more than two groups, a separate optimisation program will be solved for each group.

The first optimisation program may run the same processing operations as the second optimisation program. That is, the first and second optimisation programs may run the same optimisation algorithm as one another. Therefore, they may be the same optimisation program.

The third optimisation program may run the same processing operations as the first and second optimisation programs. That is, the first, second and third optimisation programs may run the same optimisation algorithm as one another. Therefore, they may be the same optimisation program. However, because the first and second sets of treatment parameters (as found by the first and second optimisation programs) are input into the third optimisation program, the third optimisation program may need to optimise fewer degrees of freedom than the first and second optimisation problems, and so is quicker to solve.

Each of the first optimisation program and the second optimisation program may run an optimisation algorithm which is configured to generate, as an output, treatment parameters which are optimised to achieve the treatment objectives for the treatment targets in the respective group.

The first optimisation program may be agnostic to the second group of treatment targets, and the second optimisation program is agnostic to the first group of treatment targets. That is to say, the first group may not be input into the second optimisation program, and the second group may not be input into the first optimisation program. Because the first optimisation program need only optimise treatment parameters for the first group of treatment targets, the associated optimisation time is less than it would be if it were to optimise for all of the treatment targets in the treatment volume. Furthermore, because the second optimisation program need only optimise treatment parameters for the second group of treatment targets, the associated optimisation time is less than it would be if it were to optimise for all of the treatment targets in the treatment volume.

The third optimisation program may receive as an input the first and second sets of treatment parameters, and may run an optimisation algorithm which is configured to optimise the first and second sets of treatment parameters such that the treatment objectives are achieved for the entire treatment volume, wherein the third treatment parameters comprise the optimised first and second sets of treatment parameters.

The treatment objectives may comprise one or more of: a minimum dose or a target dose for each treatment target (which target dose may be implemented by means of a dose penalisation when a dose to a treatment target is below a threshold dose, for example below a first threshold dose, such that the dose to the treatment target is promoted, e.g. increased); a maximum dose or a dose reduction for tissue (e.g. healthy tissue) surrounding the treatment targets (which dose reduction may be implemented by means of a dose penalisation when a dose to surrounding tissue is above a threshold dose, for example above a second threshold dose which is lower than the first threshold dose, such that the dose to the surrounding tissue is suppressed, e.g. decreased); a maximum dose or dose reduction for an organ at risk located in the treatment volume (which dose reduction may be implemented by means of a dose penalisation when a dose to the organ at risk is above a threshold dose, such that the dose to the organ at risk is suppressed, e.g. decreased); and optionally a maximum treatment time or a treatment time reduction (which may be implemented by means of a negative treatment time penalisation, and which may be implemented in all cases or when the treatment time is above a threshold treatment time). The target dose for the treatment targets may be defined based on a prescribed dose for delivering a clinically effective amount of radiation to the target. In exemplary embodiments, the first threshold dose may be used for each treatment target, the second threshold dose may be used for the surrounding tissue, and a maximum dose may be used for each organ at risk. A different first threshold dose may be used for different treatment targets, and a different second threshold dose may be used for different regions of the surrounding tissue. The first threshold dose(s) and the second threshold dose(s) may be soft treatment constraints which are desired but for which some flexibility is allowed under the optimisation (e.g. implemented by means of the penalisation(s)). A maximum dose and a minimum dose, on the other hand, may be hard treatment constraints which must be met by the optimisation.

The treatment parameters may comprise, for each treatment target or corresponding isocentre position, one or more of: a number of irradiations; a duration of each irradiation; a beam diameter of each irradiation; and a beam shape of each irradiation. The beam shape may be defined by collimation diameters associated with a plurality of fixed sources, or may alternatively be defined by a multi-leaf collimator (“MLC”) configuration associated with a single beam at a given location or orientation.

In some examples, the third optimisation program may only optimise a subset of the treatment parameters. For example, the third optimisation problem may only optimise a duration of each irradiation. Accordingly, by reducing the number of treatment parameters to be optimised, the third optimisation problem is computationally more simple than the first and second optimisation problems, and accordingly may be quicker to solve than if it were to optimise all treatment parameters.

In some examples, the first and second optimisation problems may optimise all treatment parameters for their respective groups, and the third optimisation problem may only optimise a subset of the treatment parameters. For example, the first and second optimisation problems may define a plurality of irradiation shots for each treatment target, wherein each irradiation shot comprises one or more irradiations each having a beam diameter, a beam shape, and an irradiation time. The third optimisation problem may then receive as an input the irradiation shots defined by the first and second optimisation problems, and may fine-tune these shots, for example by optimising a subset of the optimisation parameters, such as the irradiation time for each shot.

In other examples, the first and second optimisation problems may optimise all treatment parameters for their respective groups, and the third optimisation problem may then use the first and second sets of treatment parameters as a starting point for performing the optimisation, thereby reducing the time of the optimisation by initiating from a promising starting point.

The method may further comprise identifying a plurality of irradiation isocentre positions which map onto to the plurality of treatment targets, wherein each treatment target has at least one corresponding isocentre position; wherein the isocentre positions corresponding to the first group are input into the first optimisation program; wherein the isocentre positions corresponding to the second group are input into the second optimisation program, and wherein all of the isocentre positions are input into the third optimisation program. Herein, an isocentre position may be defined as a point in the treatment volume through which each irradiation for treating that treatment target or treatment location (for example each irradiation constituting a shot) must pass.

Where the radiotherapy system has a radiation focus point which is fixed in space, the patient may be moved relative to the focus point, such that the treatment targets or desired irradiation isocentre positions within the treatment volume coincide with the focus point of the system. For example, the patient may be moved on a support surface (e.g. bed) which moves relative to the radiotherapy system. In this case, each isocentre position may be converted into a patient treatment position in which the focus point coincides with the treatment target or the desired irradiation isocentre positions within the treatment volume.

The radiotherapy machine may be a stereotactic radiotherapy machine having a fixed focus point and a patient support moveable relative to the fixed focus point, wherein the radiotherapy machine is configured to irradiate the fixed focus point from a plurality of different incident angles and through a beam collimator having multiple collimator openings with different respective diameters, wherein the treatment parameters comprise, for each treatment target or corresponding isocentre position: a number of irradiations from each angle of incidence, a duration of each irradiation, and a collimator opening diameter for each irradiation.

The radiotherapy machine may further comprise a plurality of collimator segments, each collimator segment associated with a respective plurality of radioactive sources, and each collimator segment moveable relative to a collimator body between a plurality of collimation positions in which radiation is directed from the radioactive sources and towards the fixed focus with a predefined beam diameter, and wherein the treatment parameters comprise, for each treatment target or corresponding isocentre position: a number of radiation shots to be delivered; a collimation position of each collimator segment for each radiation shot; and a duration of each radiation shot.

In a third aspect there is provided a computer readable medium storing instructions which, when executed by a processor, cause the processor to execute a method according to any preceding aspect.

In a fourth aspect there is provided a radiotherapy system comprising at least one radiation source, a patient support moveable relative to the radiation source, and a computer readable medium according to the third aspect.

### BRIEF DESCRIPTION OF THE DRAWINGS

The present disclosure will now be described in further detail, with reference to the drawings, in which: FIG. 1a shows a collimator structure for use in a radiotherapy system according to the present disclosure;

FIG. 1b shows a cross-section of a radiotherapy system using a collimator according to FIG. 1a;

FIG. 1c shows a perspective view of the radiotherapy system from FIG. 1b;

FIG. 2 shows a radiotherapy machine according to a further example of the present disclosure;

FIG. 3 is a schematic illustration of a treatment volume grouped into clusters of treatment targets;

FIG. 4 is a flow chart showing a first preliminary method according to the present disclosure;

FIG. 5 is a flow chart showing a second preliminary method according to the present disclosure;

FIG. 6 is a flow chart showing a first method according to the present disclosure for sorting treatment targets into groups;

FIG. 7 is a flow chart showing a second method according to the present disclosure for sorting treatment targets into groups;

FIG. 8 is a flow chart showing a first method according to the present disclosure for performing optimisations on separate groups of treatment targets;

FIG. 9 is a flow chart showing a second method according to the present disclosure for performing optimisations on separate groups of treatment targets;

FIG. 10 is a schematic illustration of a first computer system according to the present disclosure; and

FIG. 11 is a schematic illustration of a second computer system according to the present disclosure.

Like reference numerals are used for like components throughout the description.

### DETAILED DESCRIPTION

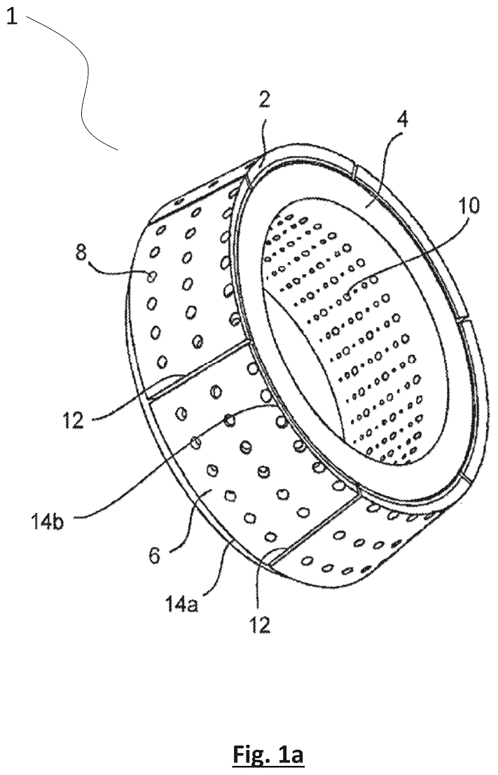

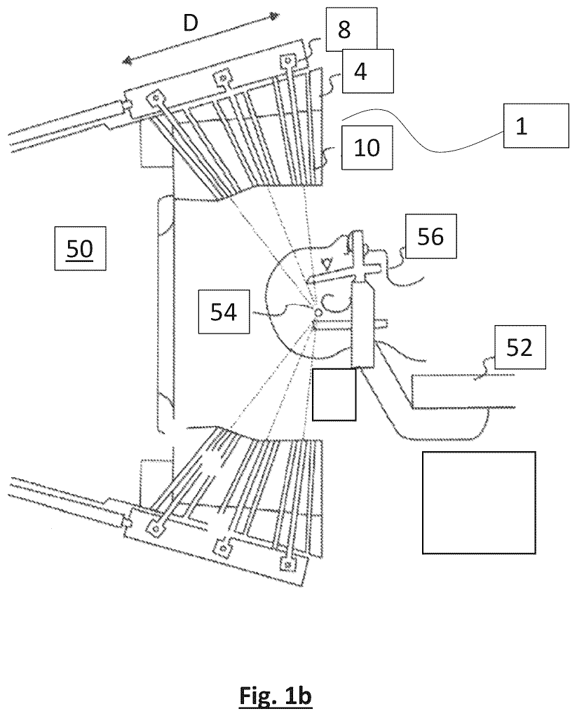

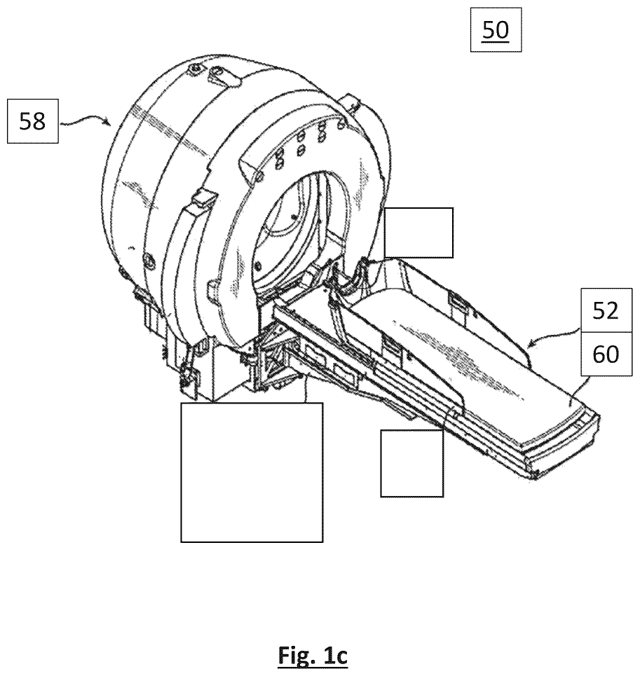

FIG. 1a shows a collimator structure 1 for use in a radiotherapy system according to the present disclosure. The collimator structure 1 has a frustoconical shape, in which the diameter of the first axial end 14a of the structure 1 is larger than the diameter of the second axial end 14b of the structure. The collimator structure comprises a collimator body 4 around which a source-carrying arrangement 2 is provided.

The source-carrying arrangement 2 comprises a plurality of openings 8. A radioactive source, for example radioactive cobalt (cobalt-60), is provided in each of the openings 8. The source-carrying arrangement 2 is split into a plurality of segments 6. In the depicted example, there are six segments. Each segment occupies a sixth of the external circumference of the collimator body 4, such that collectively the segments 6 span the entire circumference of the collimator body 4. The segments 6 meet their neighbouring segments along straight interstitial lines 12. As the reader will understand, there could be more than six segments, or fewer than six segments. Each segment is independently moveable in the fore-aft direction (perpendicular to the circumferential direction) along the collimator body, thereby moving the radioactive sources associated with the segments 6 in the fore-aft direction relative to the collimator body 4.

The collimator body 4 is formed of a radiation shielding material, for example tungsten or a tungsten alloy. The collimator body 4 includes a plurality of collimator openings 10 formed therethrough. There are three times as many collimator openings 10 as there are radioactive sources associated with the source-carrying arrangement. One third of the collimator openings 10 have a first diameter, one third of the collimator openings 10 have a second diameter which is greater than the first diameter, and the final third of the collimator openings 10 have a third diameter which is larger than the second diameter. The first collimator openings 10 may for example have a diameter of 4 mm; the second collimator openings 10 may for example have a diameter of 8 mm; and the third collimator openings 10 may for example have a diameter of 16 mm. As the reader will understand, there may be more collimator openings, for example four times as many collimator openings as the number of radioactive sources or more; or there may be fewer collimator openings, for example two times as many collimator openings as the number of radioactive sources. The diameters of the collimator openings may also be larger than or smaller than 4 mm, 8 mm, 16 mm. All of the collimator openings 10 are oriented to direct radiation through the same fixed focus point which is concentric to the collimator structure 1.

Each segment 6 may be independently moveable relative to the collimator body 4, between a first position in which the openings 8 containing the radioactive sources do not coincide with any of the collimator openings 10 and therefore are shielded by the tungsten collimator body, a second position in which the openings 8 containing the radioactive sources coincide with the first openings, a third position in which the openings 8 containing the radioactive sources coincide with the second openings, and a fourth position in which the openings 8 containing the radioactive sources coincide with the third openings. Accordingly, in the first position radiation is blocked, in the second position radiation is transmitted through the collimator body 4 with the first collimation diameter, in in the third position radiation is transmitted through the collimator body 4 with the second collimation diameter, and in the fourth position radiation is transmitted through the collimator body 4 with the third collimation diameter. Because there are six segments, each having four positions, there are 4 to the power 6 (4096) possible configurations of the collimator structure, which are individually selectable during treatment. Accordingly, the diameter of the radiation pattern received at the focus point, and the shape of the radiation pattern received at the focus point, can be selected by controlling the positions of the segments 6. Where an irradiation shot is referred to herein, that is in reference to an irradiation event in which at least one of the segments 6 is in a position other than the first position. Each shot is performed for a specific amount of time to be planned. Each isocentre may typically be irradiated with up to 5 irradiation shots, for example between 1 and 5 irradiation shots. For manual planning (not according to the present disclosure), each isocentre will generally only be irradiated with a single shot.

FIG. 1b shows a cross-section of a radiotherapy system 50 which uses a collimator structure 1 according to FIG. 1a. As shown, the collimator segments 6 are moveable in the fore-aft direction D. Furthermore, a patient support structure 52 is moveable relative to the fixed focus point 54 so that the focus point coincides with a desired treatment target within the head 56 of a patient received on the patient support structure 52.

FIG. 1c shows a perspective view of the radiotherapy system 50 from FIG. 1b. Shown in FIG. 1c is an annular housing 58 which contains the collimator structure 1; and a patient support structure 52 which includes a patient bed 60. During treatment, the bed 60 is maneuverer such that the patient is received within the annulus of the housing 58.

In the example system 1 of FIGS. 1a-1c, the treatment parameters therefore include the number of radiation shots for each treatment target/isocentre, the position of each collimator segment 6 for each irradiation shot (hereafter sometimes referred to as a collimation configuration for each shot), and the duration of each irradiation shot; and these must be defined for each treatment target/isocentre. For a given set of treatment parameters, it is possible to determine if the treatment objectives, for example the treatment constraints, are fulfilled and to calculate the penalisation(s) associated with the treatment objectives. The treatment parameters can be calculated using an optimisation problem which optimises the treatment parameters based on the desired treatment constraints and objectives.

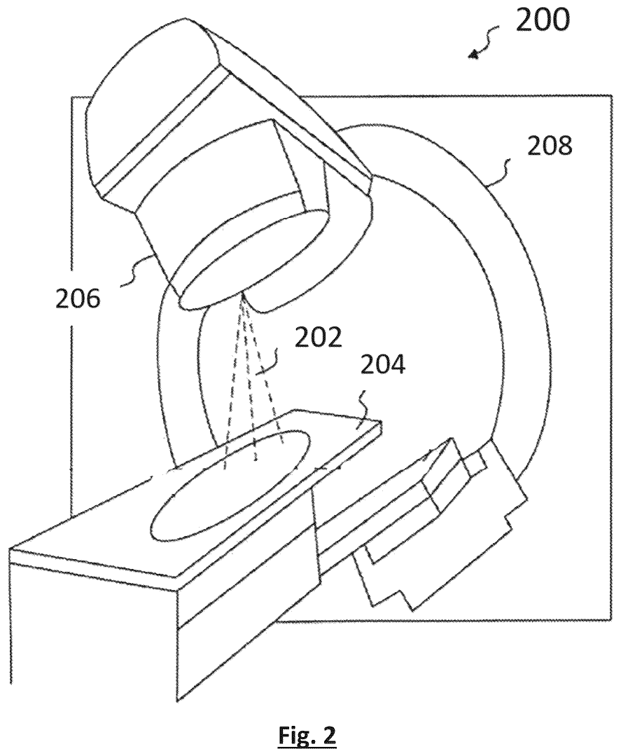

FIG. 2 shows an alternative radiotherapy system 200 according to the present disclosure. In the alternative radiotherapy system 200 according to the present disclosure, a linear accelerator (“linac”) is used to deliver radiation doses to a patient, rather than using radioactive sources and a collimator structure as in the example of FIGS. 1a-1c. The linear accelerator emits a beam 202 of radiation which is directed towards a patient arranged on a patient support bed 204. The beam 202 is emitted from a gantry head 206, which is rotatable around the bed 204 on an annular gantry structure 208. By rotating the gantry head 206 along the annular gantry structure 208, and controlling the direction of the beam 202 emitted from the gantry head 206, a plurality of different locations within a patient can be irradiated from a range of different angles.

In the example system 200 of FIG. 2, the treatment parameters include the angle of incidence for each irradiation, the beam diameter for each irradiation, the beam steering for each irradiation, the beam shape for each irradiation, and the number of irradiations; and these must be defined for each treatment target. The treatment parameters must be defined in order to achieve treatment objectives including the target radiation dose for each target, dose reduction for surrounding healthy tissue, and be below the maximum tolerance dose for any organs at risk. The treatment parameters can be calculated using an optimisation problem which optimises the treatment parameters based on the desired treatment objectives.

Therefore, in both the example system 1 of FIGS. 1a-1c, and the example system 200 of FIG. 2, it is important to carefully plan the treatment so that dosage to treatment targets tissue is maximised, and dosage to surrounding healthy tissue is minimised. This is particularly important for radiosurgical treatment of the brain, because excessive dosage to healthy tissue in the brain could have damaging neurological effects.

As has been described in the background section, one candidate use case for radio neurosurgery is the treatment of multiple brain metastases. In some cases, brain metastases can be numerous, and so many different treatment targets must be treated.

In order to effectively treat multiple brain metastases, while protecting healthy brain tissue, it is important for treatment planning to be carefully performed. In treatment planning, the treatment parameters must be carefully planned for each treatment target. As the total number of treatment targets (for example the total number of brain metastases to be treated) increases, the complexity of the optimisation problem which must be solved in order to define the treatment parameters, increases. In fact, the optimisation time increases non-linearly with the number of targets.

We will now describe the treatment planning methods according to the present disclosure which significantly reduce treatment planning time and complexity, by splitting treatment targets into groups and solving a separate optimisation problem for each group.

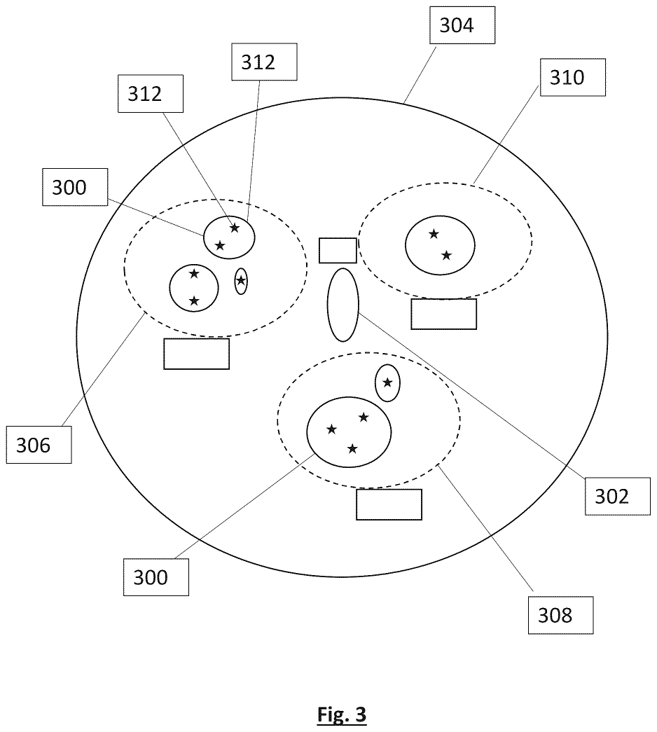

FIG. 3 shows a schematic illustration of a plurality of treatment targets 300 and an organ at risk 302 in a treatment volume 304. As shown, three of the treatment targets are clustered together in a first group 306; two of the treatment targets are clustered together in a second group 308; and a final treatment target forms a third group 310. Because the treatment targets in the first group are close to one another, they are determined to be dosimetrically significantly influencing on one another, such that a single optimisation problem must be solved for the first group. Similarly, because the treatment targets in the second group are close to one another, they are also determined to be dosimetrically significantly influencing on one another, such that a single optimisation problem must be solved for the second group. The final treatment target in the third group has no nearby treatment targets, and so forms a group of its own. Because the first, second, and third groups are far from one another, they are determined to be dosimetrically not significantly influencing one another and so can be treated in separate optimisation problems.

Also shown in FIG. 3, an organ at risk (“OAR”) 302 is located between the three groups. While it may not be accounted for in the optimisation problems for the three groups, it can be accounted for in a final pass optimisation problem once the optimisations for the three respective groups have been completed.

Because some of the treatment volumes are larger (e.g. representing larger diseased regions, such as larger cancerous metastases), they include a plurality of target isocentre locations 312 to which radiation is to be directed during treatment.

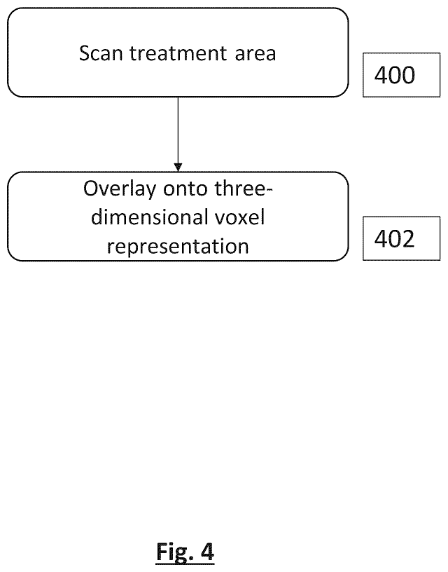

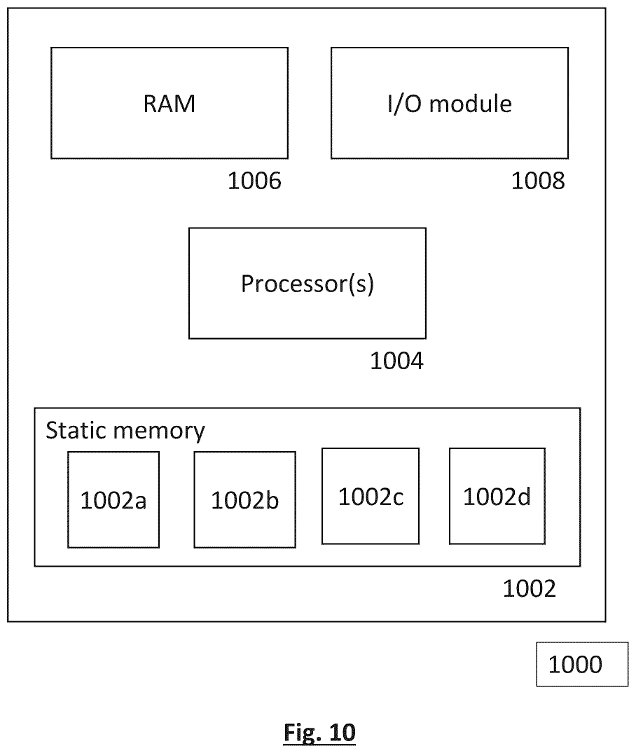

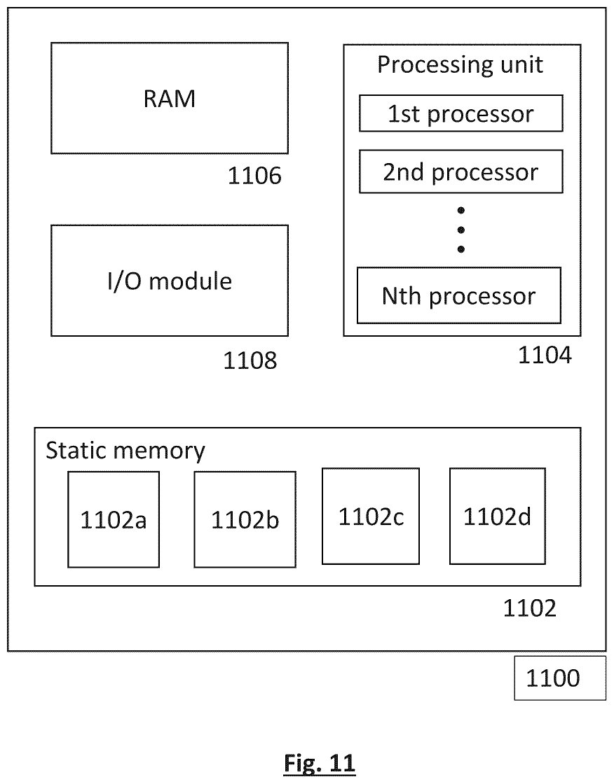

FIG. 4 shows a first preliminary method according to the present disclosure, for scanning a patient prior to performing treatment planning. The method may be performed by the computer system of FIG. 10 or of FIG. 11.

In step 400, a scan of the treatment volume (hereafter exemplified as a patient's brain) is obtained. The scan may for example be an X-ray scan, a CT scan, an MRI scan, or a PET scan. This not intended to be an exhaustive list.

In step 402, the scan is overlaid onto a three-dimensional voxel representation for the treatment volume, for which a mapping to a stereotactic frame of reference corresponding to the patient's brain is known. The mapping to the stereotactic frame of reference may be known by virtue of a stereotactic frame affixed to the patient's head (or a fiducial box attached to such a stereotactic frame), relative to which the frame of reference of the scan images (and by association the three-dimensional voxel representation) is known, or by registering an MRI or CT scan to a stereotactic volume defined by a cone-beam CT scan.

The stereotactic frame (optionally including the fiducial box) may have been attached to the patient's head prior to step 400, and may include fiducial reference markers for aligning the reference frame of the scan images.

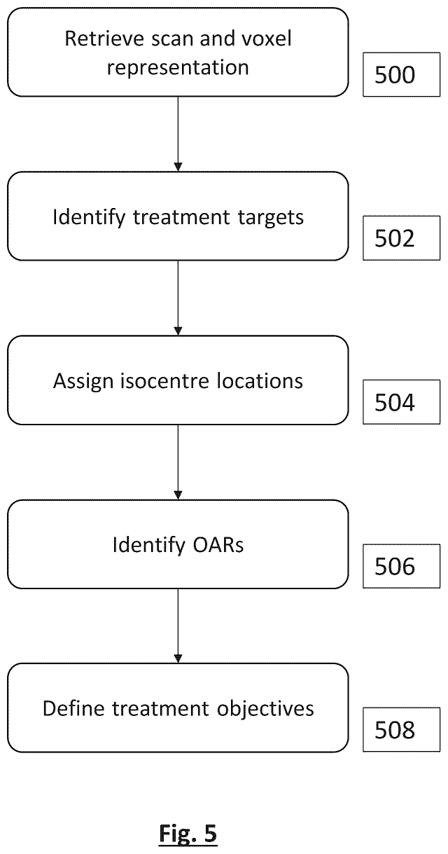

FIG. 5 shows a second preliminary method according to the present disclosure, for performing treatment planning. The second preliminary method is performed with the input of a surgeon, in order to define the treatment targets, isocentre locations, and treatment parameters, which will be used by the optimisation problems for the purpose of inverse treatment planning. The second preliminary method may be performed following the first preliminary method. The method of FIG. 5 may be performed by the computer system of FIG. 10 or of FIG. 11.

In step 500, the three-dimensional voxel representation and the scan are retrieved, and the scan is overlaid onto the three-dimensional voxel representation.

In step 502, a plurality of discrete treatment targets in the treatment volume are identified, based on the overlaid scan. For example, a plurality of discrete treatment targets may be identified in the three-dimensional voxel representation representing the treatment volume, based on the overlaid scan. A surgeon may identify the plurality of discrete treatment targets within the three-dimensional voxel representation using the overlaid scan, and input these into the computer system.

In step 504, at least one treatment isocentre location is assigned to each treatment target. For small treatment targets, only one isocentre location may be assigned. For larger treatment targets, a plurality of treatment isocentre locations may be assigned. Isocentre locations may be defined by the surgeon and input to the computer system. Alternatively, the isocentre locations may be automatically defined by the computer system based on the treatment targets as identified at step 502, and this step may be performed after step 506 or 508.

In step 506, one or more organs at risk are optionally identified, based on the overlaid scan. For example, one or more organs at risk may be identified in the three-dimensional voxel representation representing the treatment volume, based on the overlaid scan. A surgeon may identify the organ(s) at risk within the three-dimensional voxel representation using the overlaid scan, and input these into the computer system.

Finally, in step 508, treatment objectives for the treatment volume are defined. These include a target dose for each treatment target, a dose reduction (typically defined as a reduction of the dose relative to the target dose) for tissue surrounding the treatment targets, a maximum tolerance dose for any identified organ(s) at risk (or optionally a dose reduction for organ(s) at risk), and optionally a maximum treatment time or a treatment time reduction. The treatment objectives may be predefined parameters retrieved from a memory of the computer system, or may be input into the computer system by the surgeon, or may be selected by the surgeon from a list of predefined candidate treatment parameters stored in a memory of the computer system. The target dose and the dose reduction are soft constraints, in that they are desired but some flexibility is allowed under the optimisation. The maximum dose for the organs at risk is a hard constraint, in that it must be met and there is no flexibility allowed. This is described more in the “Example optimisation problem” section below.

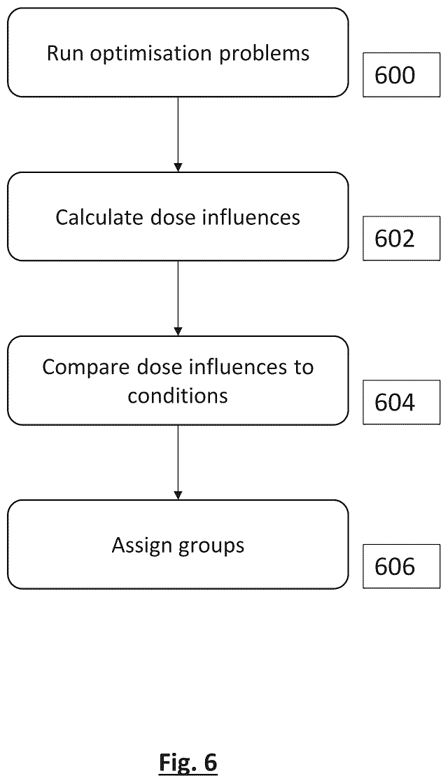

FIG. 6 shows a first method for separating the treatment targets into groups according to the present disclosure. The method of FIG. 6 may be performed by the computer system of FIG. 10 or of FIG. 11.

In step 600, each treatment target is individually input into an optimisation program (for example an optimisation program which executes/solves an optimisation problem as described more in the “Example optimisation problem” section below).

In step 602, based on the outputs from the individual optimisations for each treatment target i in step 600 (the outputs defining treatment parameters for each individual treatment target i, the treatment parameters necessarily giving rise to a radiation dose profile surrounding each individual treatment target i), a dose influence D_{i,j} from each treatment target i to each other treatment target j is determined.

In step 604, all treatment target pairs (i, j) for which the dose influence D_{i,j} from the treatment target i to treatment target j satisfies either condition [1] or condition [2] below are grouped together. By iteratively performing this process for all treatment targets in the treatment volume, the population of treatment targets in the treatment volume is sorted into groups, wherein for each group the dose influence D_{i,j} from each treatment target i in the group to at least one other treatment target j in the group satisfies condition [1] or condition [2]. Accordingly, for each group, each treatment target is considered to be dosimetrically significantly influencing on, or influenced by, at least one other treatment target in the group. Furthermore, for any pair of treatment targets selected from different respective groups, conditions [1] and [2] below are not satisfied, such that treatment targets from different groups are deemed not to be dosimetrically influencing on one another.

$${D}_{i , j} \geq θ * {D}_{presc , j} \qquad [ 1 ]$$

where θ is a predefined threshold value, where D_{presc,i} is the prescription dose for treatment target i, and where D_{presc,j} is the prescription dose for treatment target j.

In step 606, the treatment targets are assigned labels according to their assigned groups. Accordingly, the treatment targets may be grouped as illustrated in FIG. 3.

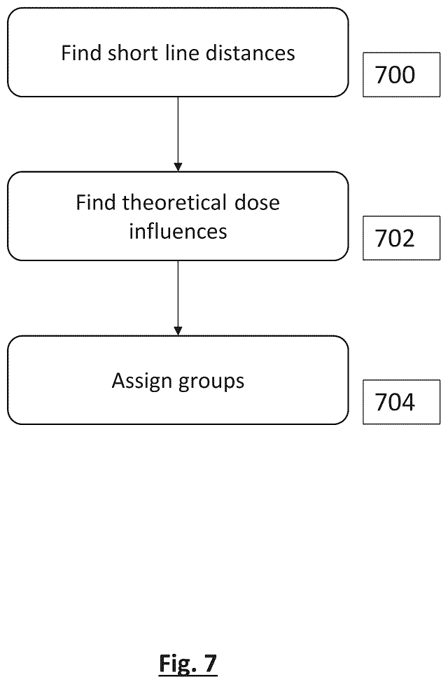

FIG. 7 shows a second method for separating the treatment targets into groups according to the present disclosure. The method of FIG. 7 may be performed by the system of FIG. 10 or of FIG. 11.

In step 700, a shortest straight-line distance Ds_{i,j} from each treatment target i to each other treatment target j is determined.

In step 702, for each pair of treatment targets (i, j), a theoretical dose received at treatment target j based on a theoretical target dose delivered to treatment target i is calculated, based on a predetermined model of dose fall-off as a function of distance and target size and/or shape. If the calculated dose received at the treatment target j is above a predetermined threshold amount, then the two treatment targets are grouped together. By repeating this process for each pair of treatment targets, the treatment targets are separated into groups, whereby each treatment target in a given group is deemed to be dosimetrically significantly influencing on, or influenced by, at least one other treatment target in the group.

In step 704, the treatment targets are assigned labels according to their assigned groups. Accordingly, the treatment targets may be grouped as illustrated in FIG. 3.

Accordingly, by employing the method of FIG. 6 or the method of FIG. 7, the treatment targets in the treatment volume are split into two or more groups of treatment targets, whereby each group contains a subset of the total number of treatment targets. Furthermore, because the groups are considered to be dosimetrically not significantly influencing on one another, optimisation problems can be solved separately for each group. Each optimisation problem is computationally less complex than solving an optimisation problem for the entire population of treatment targets. Therefore, optimisation time is reduced.

More information on the optimisation process will now be provided.

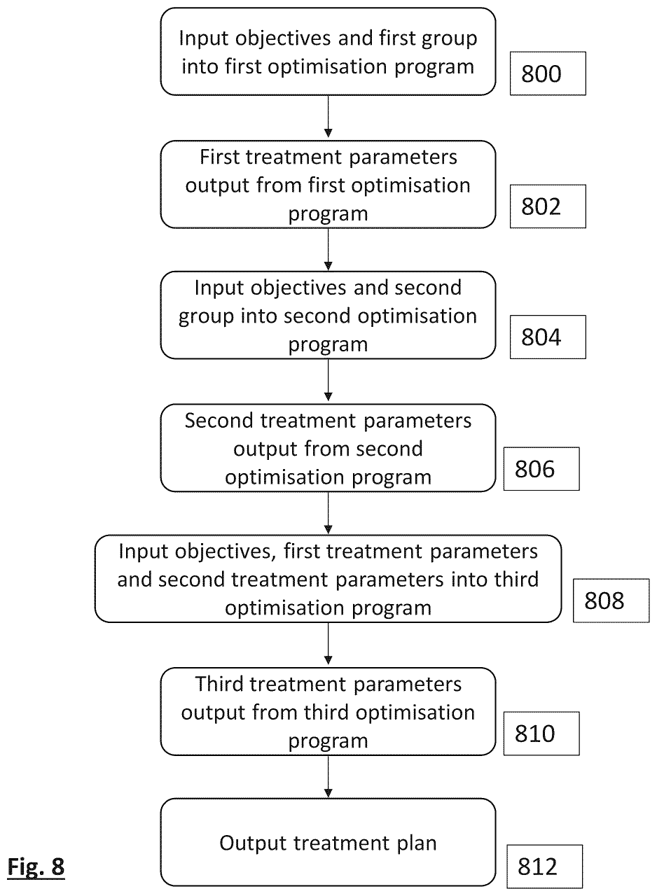

FIG. 8 shows a first method of performing optimisations for separate groups of treatment targets as determined using the method of FIG. 6 or the method of FIG. 7. The method of FIG. 8 is exemplified for two groups of treatment targets. However, as the reader will understand, the method of FIG. 8 can be performed for as many groups as are identified using the method of FIG. 6 or the method of FIG. 7. The optimisations in FIG. 8 are performed sequentially (i.e. one after the other). The method of FIG. 8 may be performed by the system of FIG. 10. The method of FIG. 8 is exemplified for the case in which the radiotherapy system is a system according to FIGS. 1a-1c. However, as the reader will understand, the optimisations of FIG. 8 could alternatively be optimisations for a system according to FIG. 2.

At step 800, the first group of treatment targets (for example as defined at step 606 or step 704) is input into an optimisation program, along with the isocentre locations associated with the treatment targets of the first group (for example as defined at step 504) and the treatment objectives for the treatment volume (for example as defined at step 508). The optimisation program used may, for example, execute/solve an optimisation problem as described more in the “Example optimisation problem” section below. In some examples, any defined OARs may also be input into the first optimisation program.

At step 802, the optimisation program solves the optimisation problem based on the inputs at step 800, and outputs a first set of treatment parameters. In particular, based on the treatment targets of the first group, the associated isocentre locations, and the treatment objectives, the optimisation algorithm generates a first set of treatment parameters which are optimised to achieve the treatment objectives for the first plurality of treatment targets and the surrounding tissue. In particular, a plurality of irradiation shots are defined, wherein the plurality of irradiation shots include at least one shot (and in some examples between 2-5 shots) for irradiating each isocentre location of the first group, and wherein the plurality of shots are collectively optimised to achieve the treatment objectives for the first plurality of treatment targets and the surrounding tissue. One or more isocentre may be assigned zero shots. Each shot is defined in terms of a collimation configuration (i.e. a position of each collimator segment 6 for the shot), and a duration of the shot. Accordingly, the first treatment parameters may comprise at least one irradiation shot corresponding to each target in the first group, wherein each shot is defined in terms of a collimation position of each segment 6, and a shot duration.

In some examples, the optimisation algorithm may be split into two phases: a segment position phase; and a sequencing phase. In the segment position phase, the optimisation algorithm may identify, for each isocentre, all positions of the respective collimator segments 6 which are required in order to appropriately irradiate the isocentre. In the sequencing phase, the required collimator segment positions may be arranged (sequenced) into a plurality of shots.

At step 804, the second group of treatment targets (for example as defined at step 606 or step 704) is input into the optimisation program, along with the isocentre locations associated with the treatment targets of the second group (for example as defined at step 504) and the treatment objectives for the treatment volume (for example as defined at step 508). The optimisation program used may, for example, execute/solve an optimisation problem as described more in the “Example optimisation problem” section below. In some examples, any defined OARs may also be input into the second optimisation program.

At step 806, the optimisation program solves the optimisation problem based on the inputs at step 804, and outputs a second set of treatment parameters. In particular, based on the treatment targets of the second group, the associated isocentre locations, and the treatment objectives, the optimisation algorithm calculates treatment parameters which are optimised to achieve the treatment objectives for the second group of treatment targets and the surrounding tissue. In particular, a plurality of irradiation shots are defined, wherein the plurality of irradiation shots include at least one shot (and in some examples between 2-5 shots) for irradiating each isocentre location of the second group, and wherein the plurality of shots are collectively optimised to achieve the treatment objectives for the second plurality of treatment targets and the surrounding tissue. One or more isocentre may be assigned zero shots. Each shot is defined in terms of a collimation configuration (i.e. a position of each collimator segment 6 for the shot), and a duration of the shot. Accordingly, the second treatment parameters may comprise at least one irradiation shot corresponding to each target in the second group, wherein each shot is defined in terms of a collimation position of each segment 6, and a shot duration.

At step 808, the first treatment parameters and the second treatment parameters are optionally input into an optimisation program (third optimisation program) which may solve the same optimisation problem as steps 800-806, and may be the same optimisation program used at steps 800-806. The treatment objectives are also input into the optimisation program. Any defined OARs from step 506 may also be input into the third optimisation program. The treatment targets from the first and second groups, and the associated isocentre locations, may also be input into the third optimisation program.

At step 810, the (third) optimisation program optionally executes an optimisation algorithm based on the inputs at step 808, and outputs a third set of treatment parameters.

In some examples, the third optimisation program uses first and second sets of treatment parameters as a ‘first guess’ for the treatment parameters for the entire treatment volume, and runs the optimisation algorithm to arrive at a third set of treatment parameters which are optimised for the entire treatment volume. This may be referred to as a warm start optimisation. In this case, by providing the first and second sets of treatment parameters as the ‘first guess’ for the third optimisation program, total optimisation time is reduced, because the starting point for the third optimisation is a promising one.

In another example, based on the first and second sets of treatment parameters, the treatment objectives, and any defined OARs, the third optimisation algorithm refines the first and second sets of treatment parameters to ensure that the treatment objectives are fulfilled for the entire treatment volume and/or for the defined OARs in the treatment volume. The third set of treatment parameters may therefore comprise a refinement of the first and second sets of treatment parameters. For example, the third optimisation problem may optimise the shot durations of the first and second sets of treatment parameters, such that the shots defined in the third set of treatment parameters are the same in number and segment collimation configuration as the first and second sets of treatment parameters, but with refined (i.e. optimised) shot durations.

At step 812, a treatment plan is output for use in radiotherapy treatment. The treatment plan includes a patient support surface 52 position for irradiation of each isocentre position (i.e. a patient support surface 52 position at which each isocentre location in the treatment volume will coincide with the fixed focus point 54 of the radiotherapy system 50). The treatment plan also includes, for each patient support surface 52 position, a number of irradiation shots, a segment 6 collimation configuration of each shot, and a shot duration of each shot.

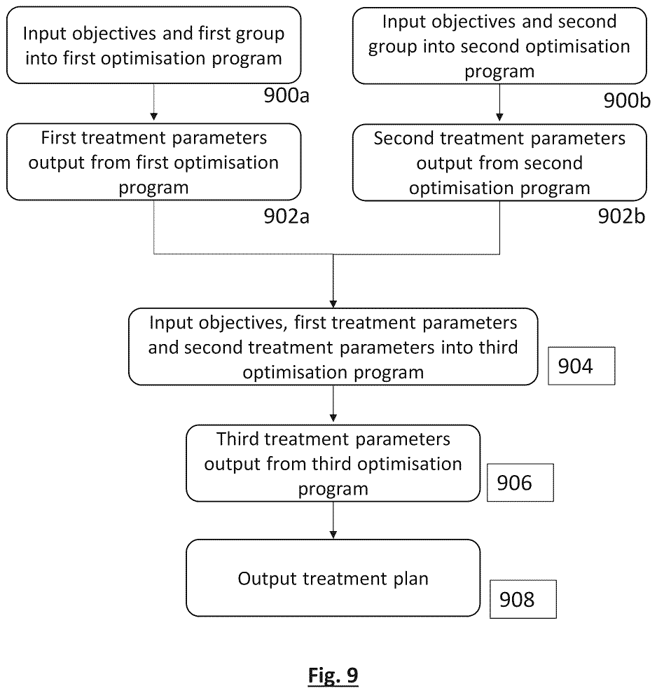

FIG. 9 shows a second method of performing optimisations for separate groups of treatment targets as determined using the method of FIG. 6 or the method of FIG. 7. The method of FIG. 9 is exemplified for two groups of treatment targets. However, as the reader will understand, the method of FIG. 9 can be performed for as many groups as are identified. The optimisations in FIG. 9 are performed in parallel (i.e. at the same time). The method of FIG. 9 may be performed by the system of FIG. 11. The method of FIG. 9 is exemplified for the case in which the radiotherapy system is a system according to FIGS. 1a-1c. However, as the reader will understand, the optimisations of FIG. 9 could alternatively be optimisations for a system according to FIG. 2.

At step 900a, the first group of treatment targets (for example as defined at step 606 or step 704) is input into an optimisation program, along with the isocentre locations associated with the treatment targets of the first group (for example as defined at step 504) and the treatment objectives for the treatment volume (for example as defined at step 508). The optimisation program used may, for example, execute/solve an optimisation problem as described more in the “Example optimisation problem” section below. In some examples, any defined OARs may also be input into the first optimisation program.

Step 900b may be performed in parallel with (i.e. concurrently with) step 900a. At step 900b, the second group of treatment targets (for example as defined at step 606 or step 704) is input into the optimisation program, along with the isocentre locations associated with the treatment targets of the second group (for example as defined at step 504) and the treatment objectives for the treatment volume (for example as defined at step 508). The optimisation program used may, for example, execute/solve an optimisation problem as described more in the “Example optimisation problem” section below. In some examples, any defined OARs may also be input into the second optimisation program.

At step 902a, the optimisation program solves an optimisation problem based on the inputs at step 900a, and outputs a first set of treatment parameters. In particular, based on the treatment targets of the first group, the associated isocentre locations, and the treatment objectives, the optimisation algorithm generates a first set of treatment parameters which are optimised to achieve the treatment objectives for the first plurality of treatment targets and the surrounding tissue. In particular, a plurality of irradiation shots are defined, wherein the plurality of irradiation shots include at least one shot for irradiating each target of the first group, and wherein the plurality of shots are collectively optimised to achieve the treatment objectives for the first plurality of treatment targets and the surrounding tissue. One or more isocentre may be assigned zero shots. Each shot is defined in terms of a collimation position of each segment 6, and a duration of the shot. Accordingly, the first treatment parameters may comprise at least one irradiation shot corresponding to each target of for the first group, wherein each shot is defined in terms of a collimation position of each segment 6, and a shot duration.

In some examples, the optimisation algorithm may be split into two phases: a segment position phase; and a sequencing phase. In the segment position phase, the optimisation algorithm may identify, for each isocentre, all positions of the respective collimator segments 6 which are required in order to appropriately irradiate the isocentre. In the sequencing phase, the required collimator segment positions may be arranged (sequenced) into a plurality of shots.

Step 902b is performed concurrently with step 902a. At step 902b, the optimisation program solves the optimisation problem based on the inputs at step 900b, and outputs a second set of treatment parameters. In particular, based on the treatment targets of the second group, the associated isocentre locations, and the treatment objectives, the optimisation algorithm calculates treatment parameters which are optimised to achieve the treatment objectives for the second group of treatment targets and the surrounding tissue. In particular, a plurality of irradiation shots are defined, wherein the plurality of irradiation shots include at least one shot for irradiating each target of the second group, and wherein the plurality of shots are collectively optimised to achieve the treatment objectives for the second plurality of treatment targets and the surrounding tissue. One or more isocentre may be assigned zero shots. Each shot is defined in terms of a collimation position of each segment, and a duration of the shot. Accordingly, the second treatment parameters may comprise at least one irradiation shot corresponding to each target of the second group, wherein each shot is defined in terms of a collimation position of each segment 6, and a shot duration.

At step 904, the first treatment parameters and the second treatment parameters are optionally input into an optimisation program (third optimisation program) which may solve the same optimisation problem as steps 900a-902b, and may be the same optimisation program used at steps 900a-902b. The treatment objectives are also input into the optimisation program. Any defined OARs from step 506 may also be input into the third optimisation program. The treatment targets from the first and second groups, and the associated isocentre locations, may also be input into the third optimisation program.

At step 906, the (third) optimisation program optionally executes an optimisation algorithm based on the inputs at step 904, and outputs a third set of treatment parameters.

In some examples, the third optimisation program uses first and second sets of treatment parameters as a ‘first guess’ for the treatment parameters for the entire treatment volume, and runs the optimisation algorithm to arrive at a third set of treatment parameters which are optimised for the entire treatment volume. This may be referred to as a warm start optimisation. In this case, by providing the first and second sets of treatment parameters as the ‘first guess’ for the third optimisation program, total optimisation time is reduced, because the starting point for the third optimisation is a promising one.

In another examples, based on the first and second sets of treatment parameters, the treatment objectives, and any defined OARs, the third optimisation algorithm refines the first and second sets of treatment parameters to ensure that the treatment objectives are fulfilled for the entire treatment volume and/or for the defined OARs in the treatment volume. The third set of treatment parameters may therefore comprise a refinement of the first and second sets of treatment parameters. For example, the third optimisation problem may optimise the shot durations of the first and second sets of treatment parameters, such that the shots defined in the third set of treatment parameters are the same in number and segment collimation configuration as the first and second sets of treatment parameters, but with refined (i.e. optimised) shot durations.

At step 908, a treatment plan is output for use in radiotherapy treatment. The treatment plan includes a patient support surface 52 position for irradiation of each isocentre position (i.e. a patient support surface 52 position at which each isocentre location in the treatment volume will coincide with the fixed focus point 54 of the radiotherapy system 50). The treatment plan also includes, for each patient support surface 52 position, a number of irradiation shots, a segment 6 collimation configuration of each shot, and a shot duration of each shot.

FIG. 10 shows a first computer system 1000 according to the present disclosure, for executing a method according to any of FIGS. 4, 5, 6, 7, 8.

The computer system 1000 comprises: a computer-readable storage medium 1002 (sometimes referred to hereafter simply as “memory” or “static memory”), such as a non-volatile computer readable storage medium, for example flash memory or a hard disc drive (HDD); one or more processors 1004; a volatile storage medium 1006, such as static random-access memory (SRAM) or dynamic random-access memory (DRAM); and an input/output (I/O) module 1008. The memory 1002 has stored thereon instructions which, when executed by the processor 1004, cause the processor to implement a method according to any of FIGS. 4, 5, 6, 7, 8. The processor 1004 is configured to execute instructions stored on the computer-readable storage medium 1002. Furthermore, the volatile storage medium 1006 is accessible by the processor 1004 in order to perform processing operations. Finally, the processor 1004 receives inputs, data and instructions from external components via the I/O module; and transmits outputs, data and instructions to external components via the I/O module. For example, the processor 1004 may receive image data, for example 3D scan data, from an external device; and may also receive surgeon-originating instructions from an external computer connected to the processor 1004 via the I/O module. The processor 1004 may also output treatment plans to the radiotherapy system via the I/O module.

As shown, stored in the memory 1002 are a scanning module 1002a comprising instructions for performing the method of FIG. 4; a preparation module 1002b comprising instructions for performing the method of FIG. 5; a grouping module 1002c comprising instructions for performing the method of FIG. 6 or FIG. 7; and an optimisation program module 1002d comprising instructions for performing the method of FIG. 8.

FIG. 11 shows a second computer system 1100 according to the present disclosure, for executing a method according to any of FIGS. 4, 5, 6, 7, 9.

The computer system 1100 comprises: a computer-readable storage medium 1102 (sometimes referred to hereafter simply as “memory” or “static memory”), such as a non-volatile computer readable storage medium, for example flash memory or a hard disc drive (HDD); a processing unit 1104; a volatile storage medium 1106, such as static random-access memory (SRAM) or dynamic random-access memory (DRAM); and an input/output (I/O) module 1108. The memory 1102 has stored thereon instructions which, when executed by the processor 1104, cause the processor to implement a method according to any of FIGS. 4, 5, 6, 7, 9. The processing unit 1104 is configured to execute instructions stored on the computer-readable storage medium 1102. Furthermore, the volatile storage medium 1106 is accessible by the processing unit 1104 in order to perform processing operations. Finally, the processing unit 1104 receives inputs, data and instructions from external components via the I/O module 1108; and transmits outputs, data and instructions to external components via the I/O module 1108. For example, the processing unit 1104 may receive image data, for example 3D scan data, from an external device via the I/O module 1108; and may also receive surgeon-originating instructions from an external computer connected to the processing unit 1104 via the I/O module 1108. The processor 1104 may also output treatment plans to the radiotherapy system via the I/O module 1108.

As shown, stored in the memory 1102 are a scanning module 1102a comprising instructions for performing the method of FIG. 4; a preparation module 1102b comprising instructions for performing the method of FIG. 5; a grouping module 1102c comprising instructions for performing the method of FIG. 6 or FIG. 7; and an optimisation program module 1102d comprising instructions for performing the method of FIG. 9.

Further, the processing unit 1104 comprises a plurality of processors. Therefore, where a plurality of optimisation programs are to be performed in parallel according to FIG. 9, each processor can run a respective optimisation program in parallel with the other processors.

### Example Optimisation Problem

An example optimisation problem according to the present disclosure will now be described in further detail. The example optimisation problem described here may be executed/solved by each of the first, second and third optimisation programs described herein.

As has been described, the inputs to the optimisation problem are: the locations of the treatment targets in the treatment volume; the isocentre locations in the treatment volume; and the treatment objectives.

Where the optimisation problem is only to optimise a single treatment target (c.f. step 600 of FIG. 6), only the single treatment target, its associated isocentre location(s), and any nearby OARs, are input into the problem. Where the optimisation is only to optimise a group representing a subset of the total number of treatment targets, only the treatment targets belonging to the group, their associated isocentre locations, and any nearby OARs, are input into the problem. Where the optimisation is to optimise all treatment targets in the treatment volume, all of the treatment targets, their associated isocentre locations, and any identified OARs in the treatment volume, are input into the problem.

The treatment objectives include a prescribed target dose for each treatment target. The prescribed target dose is defined based on a dose level which achieves the required clinical efficacy, for example the required denaturing or destruction of cancerous cells. The treatment objectives further include a dose threshold (relative to the target dose) for healthy areas of tissue surrounding and being close to the treatment targets. The dose reduction may include a dose fall-off rate (i.e. dose fall-off per unit distance) for a dose fall-off region surrounding each treatment target; and a dose reduction (defined as a reduction relative to the target dose) for tissue surrounding the dose fall-off regions. The treatment objectives may also include a maximum dose which can safely be delivered to the organ(s) at risk. The maximum dose may be a hard constraint which must be met, in order to ensure no damage to the organ(s) at risk.

The optimisation problem may assign relative weights to the treatment objectives (or these may be defined by the user as inputs to the problem). The relative weights define the relative importance of each treatment objective, relative to the other treatment objectives. An optimisation function is thereby defined, where the optimisation function is defined in terms of the treatment objectives and their respective weights. The optimisation function may include soft constraints (e.g. dose penalisations) for penalising treatment targets for which the delivered dose is below a threshold (e.g. first threshold), and for penalising surrounding tissue for which the delivered dose is above a threshold (e.g. second threshold). The optimisation function may also include hard constraints, e.g. pertaining to any organ(s) at risk. The task of the optimisation therefore becomes that of finding a set of treatment parameters for which the optimisation function is best satisfied, thereby accounting for the treatment objectives.

The optimisation problem assigns a region adjacent to each target and a dose fall-off region surrounding each treatment target. Each of these regions comprises a volumetric ring.

Again, US2018/311509, US2019/255355 and “A linear programming approach to inverse planning in Gamma Knife radiosurgery”, all of which are incorporated by reference, provide further information on optimisations of this type.

Based on the treatment objectives and the optimisation function, the optimisation problem is thereby able to define dose objectives for each of a plurality of locations throughout the treatment volume (where the plurality of locations span the treatment targets, the dose fall-off regions, the healthy tissue regions adjacent to the target, and the OAR regions). Accordingly, for each of a plurality of locations r within the treatment volume, a dose objective D(r) is defined based on the optimisation function.

The parameters to be optimised by the system are the irradiation times t_{isc} corresponding to every isocentre i, collimator segment s, and segment position c. The dose D at a given position r in the treatment volume is linear in terms of these parameters, as shown in equation [3] below, where Φ_{isc}(r) is represents predefined dose function.

$${D}_{j , i} \geq θ * {D}_{presc , i} \qquad [ 2 ]$$

In equation [3] it is assumed that the number of collimator segments and segment positions are 6 and 3, respectively. Using equation [3], it is therefore possible, for any given point (r) in the treatment volume, to calculate the dose contribution from irradiation the isocentre locations.

Based on this relationship, it is possible therefore to find the irradiation times t_{isc} corresponding to every isocentre i, collimator segment s, and segment position c for which the dose objective D(r) at each location r is best satisfied. This essentially boils down to a task of computationally finding the system parameters which best achieve a local minimum of the optimisation function. This therefore represents the optimisation which is to be performed.

The raw output from the optimisation is therefore, for each isocentre i, a time t for which each segment s is in each given position c.

As a final step, a sequencing operation is performed on the raw output, whereby the raw outputs are sequenced into a plurality of radiation shots for each isocentre, each shot being defined in terms of a collimation position of each segment and an irradiation time.

The following numbered statements set out embodiments of the disclosure:

1. A method for planning radiotherapy treatment in a treatment volume including multiple discrete treatment targets, the method comprising:
identifying the plurality of treatment targets in the treatment volume;
defining a set of treatment objectives for the treatment volume;
separating the plurality of treatment targets into at least a first group and a second group, wherein each group comprises one or more treatment targets for which a separation therebetween is below a threshold amount, and wherein each treatment target in the first group is separated from each treatment target in the second group by at least the threshold amount;
inputting the first group of treatment targets and the treatment objectives into a first optimisation program to generate a first set of treatment parameters;
inputting the second group of treatment targets and the treatment objectives into a second optimisation program to generate a second set of treatment parameters; and
inputting the first and second groups of treatment targets, the first and second sets of treatment parameters, and the treatment objectives into a third optimisation program to generate a third set of treatment parameters.

2. The method of embodiment 1, wherein the first and second optimisation programs run concurrently to generate the first and second sets of treatment parameters.

3. The method of any preceding embodiment, wherein the first optimisation program runs the same processing operations as the second optimisation program, and optionally wherein the first optimisation program is the same as the second optimisation program.

4. The method of embodiment 3, wherein each of the first optimisation program and the second optimisation program runs an optimisation algorithm which is configured to generate treatment parameters which are optimised to achieve the treatment objectives for the treatment targets in the respective group.

5. The method of any preceding embodiment, wherein the first optimisation program is agnostic to the second group of treatment targets, and wherein the second optimisation program is agnostic to the first group of treatment targets.

6. The method of any preceding embodiment, wherein the third optimisation program receives as an input the first and second sets of treatment parameters, and runs an optimisation algorithm which is configured to optimise the first and second sets of treatment parameters such that the treatment objectives are achieved for the entire treatment volume, wherein the third treatment parameters comprise the optimised first and second sets of treatment parameters.

7. The method of any preceding embodiment, further comprising identifying a plurality of isocentre positions which map onto to the plurality of treatment targets, wherein each treatment target has at least one corresponding isocentre position; wherein the isocentre positions corresponding to the first group are input into the first optimisation program;
wherein the isocentre positions corresponding to the second group are input into the second optimisation program, and wherein some or all of the isocentre positions are input into the third optimisation program, and optionally further comprising converting each isocentre position into a patient treatment position.

8. The method of any preceding embodiment, wherein the treatment objectives comprise one or more of: a minimum dose or a target dose for each treatment target; a maximum dose or a dose reduction for tissue surrounding the treatment targets; a maximum dose or a dose reduction for an organ at risk located in the treatment volume; and a maximum treatment time or a treatment time reduction.

9. The method of any preceding embodiment, wherein the treatment parameters comprise, for each treatment target or corresponding isocentre position, one or more of: a number of irradiations; a duration of each irradiation; a beam diameter of each irradiation; and a beam shape of each irradiation.

10. The method of any preceding embodiment, wherein the radiotherapy machine is a stereotactic radiotherapy machine having a fixed isocentre and a patient support moveable relative to the fixed isocentre, wherein the radiotherapy machine is configured to irradiate the fixed isocentre from a plurality of different incident angles and through a beam collimator having multiple collimator openings with different respective sizes, wherein the treatment parameters comprise, for each treatment target or corresponding isocentre position: a number of irradiations from each angle of incidence, a duration of each irradiation, and a collimator opening diameter for each irradiation.

11. The method of embodiment 10, wherein the radiotherapy machine further comprises a plurality of collimator segments, each collimator segment associated with a respective plurality of radioactive sources, and each collimator segment moveable relative to a collimator body between a plurality of collimation positions in which radiation is directed from the radioactive sources and towards the isocentre with a predefined beam diameter, and wherein the treatment parameters comprise, for each treatment target or corresponding isocentre position: a number of radiation shots to be delivered; a collimation position of each collimator segment for each radiation shot; and a duration of each radiation shot.

12. A computer readable medium storing instructions which, when executed by a processor, cause the processor to execute a method according to any preceding embodiment.

13. A radiotherapy system comprising at least one radiation source, a patient support positioned relative to the radiation source, and a computer readable medium according to embodiment 12.

1. A method for planning radiotherapy treatment in a treatment volume including multiple discrete treatment targets, the method comprising:
identifying the plurality of treatment targets in the treatment volume;
defining a set of treatment objectives for the treatment volume;
separating the plurality of treatment targets into at least a first group and a second group, wherein each group comprises one or more treatment targets for which a separation therebetween is below a threshold amount, and wherein each treatment target in the first group is separated from each treatment target in the second group by at least the threshold amount;
inputting the first group of treatment targets and the treatment objectives into a first optimisation program to generate a first set of treatment parameters;
inputting the second group of treatment targets and the treatment objectives into a second optimisation program to generate a second set of treatment parameters; and
inputting the first and second groups of treatment targets, the first and second sets of treatment parameters, and the treatment objectives into a third optimisation program to generate a third set of treatment parameters.

2. The method of claim 1, wherein the first and second optimisation programs run concurrently to generate the first and second sets of treatment parameters.

3. The method of claim 1, wherein the first optimisation program runs the same processing operations as the second optimisation program, and optionally wherein the first optimisation program is the same as the second optimisation program.

4. The method of claim 3, wherein each of the first optimisation program and the second optimisation program runs an optimisation algorithm which is configured to generate treatment parameters which are optimised to achieve the treatment objectives for the treatment targets in the respective group.

5. The method of claim 1, wherein the first optimisation program is agnostic to the second group of treatment targets, and wherein the second optimisation program is agnostic to the first group of treatment targets.

6. The method of claim 1, wherein the third optimisation program receives as an input the first and second sets of treatment parameters, and runs an optimisation algorithm which is configured to optimise the first and second sets of treatment parameters such that the treatment objectives are achieved for the entire treatment volume, wherein the third treatment parameters comprise the optimised first and second sets of treatment parameters.

7. The method of claim 1, further comprising identifying a plurality of isocentre positions which map onto to the plurality of treatment targets, wherein each treatment target has at least one corresponding isocentre position; wherein the isocentre positions corresponding to the first group are input into the first optimisation program; wherein the isocentre positions corresponding to the second group are input into the second optimisation program, and wherein some or all of the isocentre positions are input into the third optimisation program, and optionally further comprising converting each isocentre position into a patient treatment position.

8. The method of claim 1, wherein the treatment objectives comprise one or more of: a minimum dose or a target dose for each treatment target; a maximum dose or a dose reduction for tissue surrounding the treatment targets; a maximum dose or a dose reduction for an organ at risk located in the treatment volume; and a maximum treatment time or a treatment time reduction.

9. The method of claim 1, wherein the treatment parameters comprise, for each treatment target or corresponding isocentre position, one or more of: a number of irradiations; a duration of each irradiation; a beam diameter of each irradiation; and a beam shape of each irradiation.

10. The method of claim 1, wherein the radiotherapy machine is a stereotactic radiotherapy machine having a fixed isocentre and a patient support moveable relative to the fixed isocentre, wherein the radiotherapy machine is configured to irradiate the fixed isocentre from a plurality of different incident angles and through a beam collimator having multiple collimator openings with different respective sizes, wherein the treatment parameters comprise, for each treatment target or corresponding isocentre position: a number of irradiations from each angle of incidence, a duration of each irradiation, and a collimator opening diameter for each irradiation.

11. The method of claim 10, wherein the radiotherapy machine further comprises a plurality of collimator segments, each collimator segment associated with a respective plurality of radioactive sources, and each collimator segment moveable relative to a collimator body between a plurality of collimation positions in which radiation is directed from the radioactive sources and towards the isocentre with a predefined beam diameter, and wherein the treatment parameters comprise, for each treatment target or corresponding isocentre position: a number of radiation shots to be delivered; a collimation position of each collimator segment for each radiation shot; and a duration of each radiation shot.

12. A computer readable medium storing instructions which, when executed by a processor, cause the processor to:
identify the plurality of treatment targets in the treatment volume;
define a set of treatment objectives for the treatment volume;
separate the plurality of treatment targets into at least a first group and a second group, wherein each group comprises one or more treatment targets for which a separation therebetween is below a threshold amount, and wherein each treatment target in the first group is separated from each treatment target in the second group by at least the threshold amount;
input the first group of treatment targets and the treatment objectives into a first optimisation program to generate a first set of treatment parameters;
input the second group of treatment targets and the treatment objectives into a second optimisation program to generate a second set of treatment parameters; and
input the first and second groups of treatment targets, the first and second sets of treatment parameters, and the treatment objectives into a third optimisation program to generate a third set of treatment parameters

13. A radiotherapy system comprising at least one radiation source, a patient support positioned relative to the radiation source, and a computer readable medium storing instructions which, when executed by a processor, cause the processor to:
identify the plurality of treatment targets in the treatment volume;
define a set of treatment objectives for the treatment volume;
separate the plurality of treatment targets into at least a first group and a second group, wherein each group comprises one or more treatment targets for which a separation therebetween is below a threshold amount, and wherein each treatment target in the first group is separated from each treatment target in the second group by at least the threshold amount;
input the first group of treatment targets and the treatment objectives into a first optimisation program to generate a first set of treatment parameters;
input the second group of treatment targets and the treatment objectives into a second optimisation program to generate a second set of treatment parameters; and
input the first and second groups of treatment targets, the first and second sets of treatment parameters, and the treatment objectives into a third optimisation program to generate a third set of treatment parameters.
